(19) United States
(12) Patent Application Publication
(10) Pub. No.: US 2009/0191126 A1
(43) Pub. Date: Jul. 30, 2009

(54) PEPTIDE AND MULTIVALENT PEPTIDE CONJUGATE FOR DIAGNOSIS AND TREATMENT OF VASCULAR PLAQUES

(75) Inventors: Claude F. Meares, Davis, CA (US); Mark R. McCoy, Davis, CA (US); Jeffrey J. Day, Davis, CA (US); Jinsheng Li, Duluth, GA (US)

Correspondence Address: MORGAN, LEWIS & BOCKIUS LLP (SF), One Market, Spear Street Tower, Suite 2800, San Francisco, CA, 94105, US

(73) Assignees: THE REGENTS OF THE UNIVERSITY OF CALIFORNIA, Oakland, CA (US); CLEAR VASCULAR, INC., New York, NY (US)

(21) Appl. No.: 12/325,110

(22) Filed: Nov. 28, 2008

Related U.S. Application Data
(60) Provisional application No. 60/990,549, filed on Nov. 27, 2007.

(51) Int. Cl. A61K 49/00; C07K 7/06; C07K 7/64; A61K 38/08

(52) U.S. Cl. 424/9.1; 530/328; 530/317; 514/15

(57) ABSTRACT

This invention encompasses compositions and methods for treating and imaging vulnerable plaque and other inflamed regions in a subject.

### CROSS-REFERENCE TO RELATED APPLICATIONS

The present application claims priority under 35 U.S.C. §119(e) to U.S. Provisional Patent Application No. 60/990,549 filed Nov. 27, 2007, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No. CA16861, awarded by the NIH/NCI to C. F. Meares. The Government has certain rights in this invention.

### FIELD OF THE INVENTION

The present invention relates generally to medical methods and compositions. More particularly, the present invention relates to methods and compositions for treating and imaging regions of inflammation in body lumens such as vulnerable plaque in the vasculature.

### BACKGROUND OF THE INVENTION

Coronary artery disease resulting from the build-up of atherosclerotic plaque in the coronary arteries is a leading cause of death in the United States and worldwide. The plaque build-up causes a narrowing of the artery, commonly referred to as a lesion, which reduces blood flow to the myocardium (heart muscle tissue). Myocardial infarction (better known as a heart attack) can occur when an arterial lesion abruptly closes the vessel, causing complete cessation of blood flow to portions of the myocardium. Even if abrupt closure does not occur, blood flow may decrease resulting in chronically insufficient blood flow which can cause significant tissue damage over time.

A variety of interventions have been proposed to treat coronary artery disease. For disseminated disease, the most effective treatment is usually coronary artery bypass grafting where problematic lesions in the coronary arteries are bypassed using external grafts. In cases of less severe disease, pharmaceutical treatment is often sufficient. Finally, focal disease can often be treated intravascularly using a variety of catheter-based approaches, such as balloon angioplasty, atherectomy, radiation treatment, stenting, and often combinations of these approaches.

With the variety of diagnostic and treatment techniques which are available, the cardiologist is faced with a challenge of selecting the particular treatment which is best suited for an individual patient. While numerous diagnostic aids have been developed, no one technique provides all the information which is needed to select a treatment. Angiography is very effective in locating lesions in the coronary vasculature, but provides little information concerning the nature of the lesion. This is especially true for vulnerable plaque, which resides primarily outside the vessel lumen and is generally not detectable by angiography. To provide better characterization of the lesion(s), a variety of imaging techniques have been developed for providing a more detailed view of the lesion, including intravascular ultrasound (IVUS), angioscopy, laser spectroscopy, computed tomography (CT), magnetic resonance imaging (MRI), and the like. None of these techniques, however, is completely successful in determining the exact nature of the lesion. In particular, such techniques provide little information regarding whether the plaque is stable or unstable.

Plaques which form in the coronaries and other vessels comprise inflammatory cells, smooth muscles cells, cholesterol, and fatty substances, and these materials are usually trapped between the endothelium of the vessel and the underlying smooth muscle cells. Depending on various factors, including thickness, composition, and size of the deposited materials, the plaques can be characterized as stable or unstable. The plaque is normally covered by an endothelial layer. When the endothelial layer is disrupted, the ruptured plaque releases highly thrombogenic constituent materials which are capable of activating the clotting cascade and inducing rapid and substantial coronary thrombosis. Such rupture of an unstable plaque and the resulting thrombus formation can cause unstable angina chest pain, acute myocardial infarction (heart attack), sudden coronary death, and stroke. It has recently been proposed that plaque instability, rather than the degree of plaque build-up, should be the primary determining factor for treatment selection.

A variety of approaches for distinguishing stable and unstable plaque in subjects is are proposed. Some of the proposals involve detecting a slightly elevated temperature within unstable plaque resulting from inflammation. Other techniques involve exposure of the plaque to infrared light. It has also been proposed to introduce radio labeled materials which have been shown by autoradiography to bind to stable and unstable plaque in different ways. External detection of the radiolabels, however, has limited the sensitivity of these techniques and makes it difficult to determine the precise locations of the affected regions. It would therefore be of great benefit to provide for improved radiolabels, compositions, and protocols for detecting vulnerable plaque and other inflammatory luminal lesions by specifically targeting those lesions.

Once vulnerable plaque has been detected, it would be of significant benefit to provide methods for treating that plaque to reduce the risk of rupture. Conventional intravascular treatments for stenotic lesions, such as angioplasty, atherectomy, and stenting may have only limited value in treating vulnerable plaques and in some instances might actually induce acute thrombosis at the site of the vulnerable plaque. Thus, it would be desirable to provide methods and compositions for treating vulnerable plaque to lessen the risk of rupture and abrupt closure.

### SUMMARY OF THE INVENTION

The present invention provides compositions and methods for treating and/or imaging regions of apoptotic tissue, vulnerable plaque, inflammatory conditions and the like within a blood vessel or other body lumen of a subject. While the invention is illustrated by reference to treating vulnerable plaque within a subject's vascular system, particularly the arterial system, including the coronary, peripheral, and cerebral arterial systems, it will be appreciated the invention is also useful for treating various conditions, e.g., inflammatory conditions, in addition to vulnerable plaque and treating body lumens and other target sites in addition to the vasculature

In an exemplary embodiment, a diseased tissue, e.g., a lumen having vulnerable plaque, inflammatory conditions, etc., is treated by administering a metal chelate to a subject. In an exemplary embodiment the metal chaleta is a conversion electron emitting source (CEES). The CEES is preferably a metal, e.g. Sn-117m, holmium-I 66, thallium-201, technetium-99m, or the like. For exemplary therapeutic purposes, the CEES is administered at a dose sufficient to inhibit rupture of vulnerable plaque, and/or treat vulnerable plaque which has ruptured. Any useful dosage range and dosing regimen may be used. In an exemplary embodiment, a typical total dosage range is from about 0.05 microcuries to about 20 millicuries, more preferably in the range from about 0.5 millicurie to about 10 millicurie. For exemplary imaging applications, the CEES is delivered under conditions which allow it to localize at a target tissue, e.g., a region of vulnerable plaque, inflammatory response, etc., and imaging is based on external or other detection of emitted gamma radiation.

In certain embodiments, the peptides or proteins of the present invention are attached to imaging agents of use for imaging and diagnosis of disease states in various organs, tissues or cell types. Many appropriate imaging agents are known in the art, as are methods for their attachment to proteins or peptides (see, e.g., U.S. Pat. Nos. 5,021,236 and 4,472,509). The art is replete with methodologies for the attachment of metal chelates to targeting species. Exemplary methods involve the use of a metal chelate complex employing, for example, an organic chelating agent such as DTPA attached to the protein or peptide (U.S. Pat. No. 4,472,509).

Other objects, advantages, and embodiments of the invention are set forth in the detailed description that follows.

### BRIEF DESCRIPTION OF THE DRAWINGS

FIG. 1A & FIG. 1B: Pictures of aorta containing rabbit vulnerable plaque from the RVP C7×C A (A) and from the negative control (B).

FIG. 2: Peptide CTPHTNQTC (SEQ ID NO: 1) chemical structure.

FIG. 3: Amplifier molecule chemical structure.

FIG. 4: Dendrimer comprising the peptide CTPHTNQTC (SEQ ID NO: 1).

### DETAILED DESCRIPTION OF THE INVENTION AND THE PREFERRED EMBODIMENTS

### Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, See, e.g., Matthews, PLANT VIROLOGY, 3rd edition (1991); Sambrook, Fritsch and Maniatis, MOLECULAR CLONING: A LABORATORY MANUAL, 2nd edition (1989); CURRENT PROTOCOLS 1N MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987), each of which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

As used in the specification and claims, the singular form “a”, “an” and “the” include plural references unless the context clearly dictates otherwise. For example, the term “a cell” includes a plurality of cells, including mixtures thereof.

Peptides may be synthesized using solid phase technology. The principles of solid phase chemical synthesis of polypeptides are well known in the art and may be found in general texts relating to this area (Dugas, H. and Penney, C. 1981 Bioorganic Chemistry, pp 54-92, Springer-Verlag, New York). Wild type and artificial proteins and polypeptides can be synthesized by solid-phase methodology utilizing an Applied Biosystems 430A peptide synthesizer (Applied Biosystems, Foster City, Calif.) and synthesis cycles supplied by Applied Biosystems. Protected amino acids, such as t-butoxycarbonyl-protected amino acids, and other reagents are commercially available from many chemical supply houses.

“Peptide,” “polypeptide” or “protein” refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alamine, phenylglycine and homoarginine are also included. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids, thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups alos include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

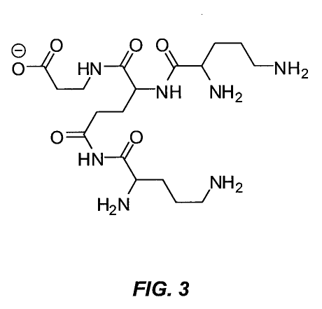

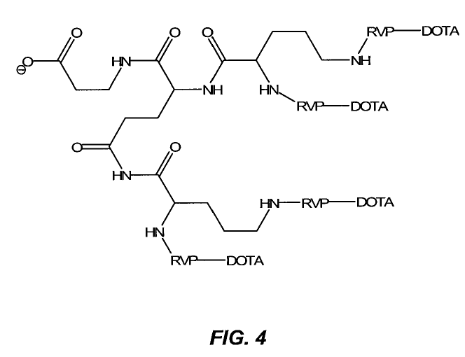

The term “targeting moiety” is intended to mean a moiety that is (1) able to direct the entity to which it is attached (e.g., therapeutic agent or marker) to a target cell, for example to apoptotic tissue or (2) is preferentially activated at a target tissue, for example apoptotic tissue (e.g. atherosclerotic tissue), for example an atherosclerotic plaque. The targeting group can be a small molecule, which is intended to include both non-peptides and peptides. In an exemplary embodiment the small molecule can have a molecular weight greater than 1,000. In certain embodiments, the small molecule is polyvalent, with a central core bound to one or more peptides. In various embodiments, two copies of a peptide are bound to the central core. In certain embodiments up to ten copies of a peptide are bound to the central core. In an exemplary embodiment the central core can be a dendrimer. An exemplary embodiment includes a polyvalent peptide. An exemplary embodiment includes the chemical structure of FIG. 3 and FIG. 4. The targeting group can also be selected from saccharides, lectins, receptors, ligand for receptors, proteins such as BSA, antibodies, peptides, synthetic peptides, polyvalent peptides, and so forth. In an exemplary embodiment, the targeting moiety, or portion thereof, is bound to a metal chelate

The term “binding domain” is intended to mean a moiety that binds a therapeutic or imaging agent. In an exemplary embodiment the binding domain includes an antibody. In an exemplary embodiment the binding domain is an antibody that binds a metal chelate. In an exemplary embodiment, the metal chelate is a macrocyclic metal chelate. In an exemplary embodiment, the metal chelate is a member selected from substituted or unsubstituted EDTA and substituted or unsubstituted DOTA, substituted or unsubstituted NOTA (triazacyclononane triacetic acid). In another exemplary embodiment, the metal chelate is a member selected from substituted or unsubstituted AABD, substituted or unsubstituted BAD, substituted or unsubstituted ABD, substituted or unsubstituted NBD and substituted or unsubstituted sulfhydryl DOTA. In various embodiments the metal chelate further comprises a reactive functional group. In an embodiment the reactive functional group has complementary reactivity to a cysteine substitution on an antibody. In several embodiments the metal chelate is bound to an antibody via the cysteine substitutions.

In one embodiment, the targeting moiety is covalently attached to a binding domain. In an embodiment the targeting moiety includes a molecule that binds apoptotic tissue. In various embodiments the targeting moiety includes a molecule that specifically binds atherosclerotic plaques. In certain embodiments, the targeting moiety includes a molecule that selectively binds proteins that are over-expressed in apoptotic tissue. In an exemplary embodiment the targeting moiety binds perilipin.

In various exemplary embodiments, the targeting moiety includes the peptide CTPHTNQTC (SEQ ID NO: 1). In an exemplary embodiment, the targeting moiety includes an amplifier (e.g. a dendrimer) functionalized with the peptide CTPHTNQTC (SEQ ID NO: 1).

The term “amplifier” is intended to mean a multifunctional group or backbone providing a plurality of attachment sites. Oligomers and polymers, including polypeptides, polysaccharides and others, are generally useful for this backbone. In one embodiment the amplifier includes a dendrimeric component. In various exemplary embodiments, the amplifier is the molecule

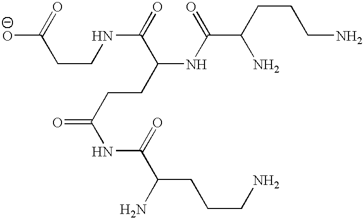

In an exemplary embodiment, the amplifier is covalently attached to the peptide CTPHTNQTC (SEQ ID NO: 1).

As used herein, the term “dendrimer” is used as this term is generally used in the art. The term refers to dendritic structures such as dendronized polymers, dendritic stars, dendritic linear hybrids, and the like. Dendrimers can include polymers of spherical or other three-dimensional shapes that have precisely defined compositions and that possess a precisely defined molecular weight. Dendrimers can be synthesized as water-soluble macromolecules through appropriate selection of internal and external moieties. See, U.S. Pat. Nos. 4,507,466 and 4,568,737. Since the synthesis and characterization of the first dendrimers, a large array of dendrimers of diverse sizes and compositions has been prepared. See, for example, Newkome, G R, Moorefield, C N and Voegtle, F. “Dendritic Molecules” VCH, Weinheim, 1996; and Liu M. and Frechet J. M. J., Pharm. Sci. Tech. Today 2(11):393 (1999). Dendritic macromolecules are characterized by a highly branched, layered structure with a multitude of chain ends. Dendrimers are particularly well defined with a very regular and almost size monodisperse structure, while hyperbranched polymers are less well defined and have a broader polydispersity. Dendrimers have been conjugated with various pharmaceutical materials as well as with various targeting molecules that may function to direct the conjugates to selected body locations for diagnostic or therapeutic applications. See, for example, WO 8801178, see also a review by Liu M. and Frechet J. M. J., Pharm. Sci. Tech. Today 2(11):393 (1999). Dendrimers have been used to covalently couple synthetic porphyrins (e.g., hemes, chlorophyll) to antibody molecules as a means for increasing the specific activity of radiolabeled antibodies for tumor therapy and diagnosis. Roberts et al., Bioconjug. Chemistry 1:305 308 (1990); Tomalia et al., U.S. Pat. No. 5,714,166. In one embodiment the dendrimer is an alkyl. In certain embodiments the denderimer is a heteroalkyl. In various embodiments the dendrimer is an aryl.

The term “apoptotic tissue” is intended to mean tissue wherein the cells are undergoing apoptosis. Apoptosis refers to “programmed cell death” whereby the cell executes a “cell suicide” program. The consequences of undesired apoptosis can be devastating pathologies such as including ischemic injury, such as typically occurs in cases of myocardial infarction, reperfusion injury and stroke. Apoptosis within atherosclerotic plaques can be associated with plaque vulnerability and rupture.

As used herein, “therapeutic agent” means any agent useful for therapy including cytotoxins, and radioactive agents.

As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used herein, “a radioactive agent” includes any radioisotope, which is effective in treating atherosclerotic plaque. Examples include, but are not limited to, Sn-117m, indium-111, Y-90, Lu-177, Sm-153, Er-169, Dy-165, Cu-67, cobalt-60 and X-rays. Additionally, naturally occurring radioactive elements such as uranium, radium, and thorium, which typically represent mixtures of radioisotopes, are suitable examples of a radioactive agent.

As used herein, “administering” means oral administration, intranasal administration, administration as a suppository, topical contact, intravenous, intraperitoneal, intramuscular, intralesional or subcutaneous administration, or the implantation of a slow-release device e.g., a miniosmotic pump, to the subject.

As used herein, “cell surface antigens” means any cell surface antigen which is generally associated with cells involved in a pathology (e.g., apoptotic cells, apoptotic tissue antigens, atherosclerotic plaque antigens), i.e., occurring to a greater extent as compared with normal cells. Such antigens may be tissue specific. Alternatively, such antigens may be found on the cell surface of both diseased and non-diseased cells. These antigens need not be specific to diseased tissue. However, they are generally more frequently associated with diseased tissue than they are associated with normal tissue. An exemplary embodiment of a cell surface antigen is perilipin.

As used herein, “pharmaceutically acceptable carrier” includes any material which when combined with the compositions of the current invention and retain the compositions functionality. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, the term “isolated” means separated from constituents, cellular and otherwise, in which a polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated,” “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart.

“Recombinant” as applied to a polynucleotide means that the polynucleotide is the product of various combinations of cloning, restriction and/or ligation steps, and other procedures that result in a construct that is distinct from a polynucleotide found in nature.

In various embodiments the present invention provides for peptides that target therapeutic and imaging agents to diseased or injured tissues, organs and/or cells for therapeutic and diagnostic purposes. In various embodiments the agent is a metal chelate. In an exemplary embodiment, the metal chelate is a macrocyclic metal chelate. In various exemplary embodiments, the metal chelate is a member selected from substituted or unsubstituted EDTA, substituted or unsubstituted DTPA, substituted or unsubstituted TETA and substituted or unsubstituted DOTA.

In an exemplary embodiment, the agent includes a metal that is a conversion electron emitting source (CEES). The chelating agent complexed to the CEES can be modified or configured to enhance localization at regions of diseased tissue, e.g. apoptotic tissue, vulnerable plaque or other inflammatory regions. Pharmaceutical therapeutic compositions according to various embodiments of the present invention can be administered to a subject, including humans and animals, by parenteral, systemic, or local injections into vasculature or other locations, including the epidural, the subarachnoid compartment, solid tissue, the pulmonary system, the reticuloendothelial system, potential cavities, and the like. The compositions and methods are suitable for imaging atherosclerotic atheroma, commonly referred to as hard plaque, as well as soft or vulnerable plaque, although treatment is particularly effective for the soft or vulnerable plaque.

Imaging can include any mode of detecting localized CEESs, including the detection of gamma photon emission from the CEESs.

In various embodiments, the CEES is tin, e.g., Sn-117m, which primarily emits conversion electrons and gamma photons. In various embodiments, the metal used is selected from holmium-I 66, thallium-20 I, and technetium-99m. Sn-117m is preferably in metallic form. Methods of forming Sn-117m are known in the art and include preparation in an accelerator, such as a linear accelerator or a cyclotron, by, for example, transmutation of antimony into known “No-Carrier-Added” Sn-117m by intermediate to high energy proton induced reactions. Alternatively, thermal or fast neutron bombardment of Sn-117m or several other elements, using uranium-235, uranium-233, or plutonium-239, can be performed in a reactor to produce tin-117m and other CEESs appropriate for the applications and compositions disclosed herein. The production of Sn-117m is well known in the art.

In various compositions of the present invention, the Sn-117m or other CEES is coupled, attached, or otherwise bound to a peptide which preferentially or specifically binds to a component of diseased tissue, e.g. apoptotic tissue, a vulnerable plaque or other inflammatory site for diagnostic or therapeutic purposes. The Sn-117m is generally complexed with a metal chelating agent, which is itself derivatized to facilitate its attachment to a targeting moiety.

In a first aspect, the invention includes a purified peptide that selectively binds to atherosclerotic plaque. The peptide comprises the amino acid sequence CTPHTNQTC (SEQ ID NO: 1). In an exemplary embodiment the peptide has the chemical structure:

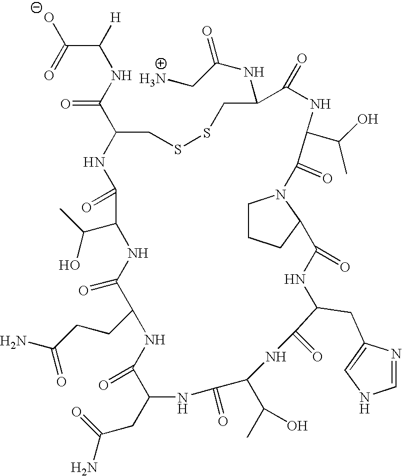

In an exemplary embodiment, the peptide serves as a targeting moiety and is covalently bound to a metal chelate. In various embodiments, the metal chelate is a macrocyclic metal chelate. In an exemplary embodiment, the metal chelate is a member selected from substituted or unsubstituted EDTA, substituted or unsubstituted DTPA, substituted or unsubstituted TETA and substituted or unsubstituted DOTA. In an exemplary embodiment, the targeting moiety, or portion thereof, is covalently bound to the metal chelate.

In various embodiments, the peptide has the formula X-(RVP)_{N}-M, wherein X is an amplifier molecule, RVP is a peptide comprising the amino acid sequence CTPHTNQTC (SEQ ID NO: 1), N is an integer from 2-100, and M is an agent selected from the group consisting of a therapeutic agent and an imaging agent. In several embodiments the amplifier is a dendrimer. In several embodiments the amplifier comprises dendritic structures such as dendronized polymers, dendritic stars, and dendritic linear hybrids. In certain embodiments, the amplifier molecule has the chemical structure:

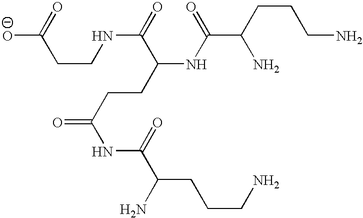

In an exemplary embodiment the peptide has the chemical structure:

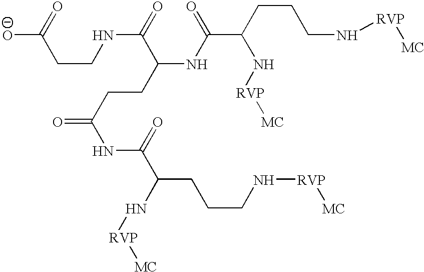

wherein RVP comprises the amino acid sequence CTPHTNQTC (SEQ ID NO: 1) and MC represents a metal chelate, as discussed herein.

In certain embodiments, the peptide comprises tin-117m complexed thereto. In an exemplary embodiment the tin chelate is a member of the group consisting of Sn(II) and Sn(IV). In various embodiments the peptide comprises a pharmaceutical composition comprising the peptide and a pharmaceutically acceptable carrier.

In an exemplary embodiment, the peptide is bound to a binding moiety for a metal chelate. In various embodiments the binding moiety is an antibody. In various embodiments the antibody is naturally occurring. In an exemplary embodiment the antibody is recombinantly expressed. Both whole antibodies and fragments are of use in the invention. In several embodiments the antibody is a scFv. In various embodiments the antibody is specific for a metal chelate. In an exemplary embodiment the antibody is specific for substituted or unsubstituted DOTA. In an exemplary embodiment the metal chelate is covalently attached to the antibody. In an exemplary embodiment the antibody is 2D12.5. Methods to design and recombinantly express antibodies and antibody fragments which recognize and bind to metal chelates are known in the art. In an exemplary embodiment the peptide comprises a dendrimer covalently attached to an antibody.

When components of an agent of the invention are to be conjugated together to form a multi-component agent (e.g., metal chelate-antibody, metal chelate peptide, peptide-antibody, etc.) the precursors of the components of the agent bear at least one reactive functional group, which can be located at any position on the component. The components are covalently bonded through reaction of reactive functional groups of complimentary reactivity on the component precursors. Reactive functional groups and classes of reactions useful in practicing the present invention are generally those that are well known in the art of bioconjugate chemistry. Currently favored classes of reactions available with reactive agent components are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be, for example, converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

The reactive functional groups can be chosen such that they do not participate in, or interfere with, the reactions necessary to assemble a component of the agent, e.g., the chelate, peptide, etc., or the agent itself. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

The components of the agents of the invention, e.g., the chelate and the peptide, the peptide and the antibody, etc., can be joined by a linker. Linkers of use in the present invention include those of zero-order as well as substituted or unsubstituted alkyl, substituted or unsubstituted heteroalkyl, substituted or unsubstituted cylcoalkyl, substituted or unsubstituted aryl, and substituted or unsubstituted heteroaryl moieties that include at least two reactive functional groups through which the components of the agent can be attached to the linker. Thus, preferred linker precursors are bi-, tri, tetra-, and penta-functionalized with reactive functional groups.

In one embodiment, the linker attaches the components of the ligand to each other essentially irreversibly via a “stable bond” between the components. A “stable bond”, as used herein, is a bond, which maintains its chemical integrity over a wide range of conditions (e.g., amide, carbamate, carbon-carbon, ether, etc.). In another embodiment the linker attaches two or more chelate components by a “cleaveable bond”. A “cleaveable bond”, as used herein, is a bond which is designed to undergo scission under selected conditions. Cleaveable bonds include, but are not limited to, disulfide, imine, carbonate and ester bonds.

### A. The Methods

In addition to the compositions of the invention, in another aspect, the invention also provides methods of using the compositions of the invention to image diseased and injured tissue, e.g., apoptotic tissue, vulnerable plaque and inflammation in a subject.

Thus, in one aspect, the invention provides a method of using the compositions of the invention to image diseased tissue in a subject. In an exemplary embodiment, the diseased tissue is an atherosclerotic plaque, e.g., a vulnerable plaque. In an exemplary embodiment the diseased tissue is apoptotic and/or inflamed. In an exemplary embodiment a peptide is used to image the diseased tissue. In various embodiments the peptide CTPHTNQTC (SEQ ID NO: 1) is used to image the diseased tissue. In various embodiments the peptide is covalently attached to a metal chelate. In an exemplary embodiment the peptide is attached to the metal chelate Sn-117m. In various embodiments the peptide is comprised of a dendrimer.

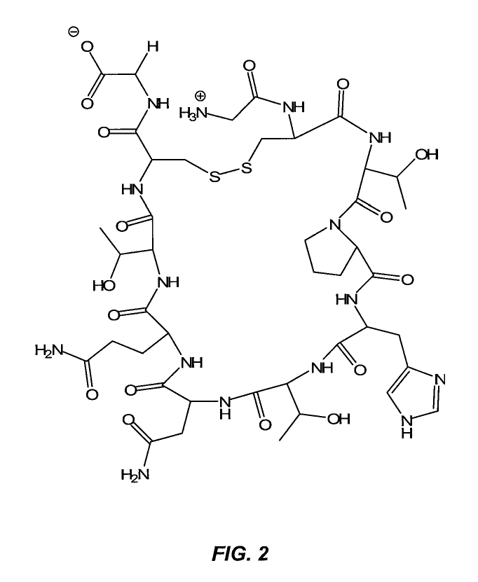

In various embodiments the invention comprises using an agent that comprises a peptide with the chemical structure of FIG. 2.

In various embodiments the invention comprises using an agent that comprises the formula X-(RVP)_{N}-M wherein X is an amplifier molecule, RVP is a peptide comprising the amino acid sequence CTPHTNQTC (SEQ ID NO: 1), N an integer from 2-100, and M is an agent selected from the group consisting of therapeutic agent and imaging agent according to any of the preceding paragraphs.

In various embodiments the invention comprises using an agent that comprises an amplifier molecule that has the chemical structure of FIG. 3.

In various embodiments the invention comprises using an agent that comprises an amplifier molecule that has the chemical structure of FIG. 4. wherein RVP comprises the amino acid sequence CTPHTNQTC (SEQ ID NO: 1) and MC comprises a metal chelate according to any of the preceding paragraphs.

In another aspect, the invention provides methods of using the compositions of the invention to treat atherosclerotic plaques in a subject. An exemplary method for inhibiting inflammation in hyperplasia in body lumens and other body target sites includes delivering or implanting a CEES attached to a peptide that selectively binds to atherosclerotic plaques, to or within the body lumen or other body site. Methods are particularly useful for treating vulnerable plaque in the vasculature. Hyperplasia and inflammation, however, can also affect other body lumens, including the ureter, urethra, arterial venous dialysis shunts, the vaginal canal, the cervical os, the esophagus, the trachea, the bronchioles, the bronchi, and gastrointestinal tract, ostomies, biliary and pancreatic ducts, and the like. The method includes administering to the subject a purified peptide that selectively binds to the diseased tissue. Thus, in one aspect, the invention provides a method of using the compositions of the invention to treat a subject for a disease or condition (e.g., atherosclerosis) or to diagnose a condition or disease, the method comprising the steps of: (a) administering to the subject a purified peptide that selectively binds to atherosclerotic plaque wherein the peptide comprises the amino acid sequence CTPHTNQTC (SEQ ID NO: 1), wherein the peptide is covalently bound to an agent selected from the group consisting of therapeutic agent and imaging agent, thereby forming with the apoptotic tissue an apoptotic tissue-peptide-agent complex; and (b) treating the condition.

In various embodiments the invention comprises using an agent that comprises a peptide with the chemical structure of FIG. 2.

In various embodiments the invention comprises using an agent that comprises the formula X-(RVP)_{N}-M wherein X is an amplifier molecule, RVP is a peptide comprising the amino acid sequence CTPHTNQTC (SEQ ID NO: 1), N an integer from 2-100, and M is an agent selected from the group consisting of therapeutic agent and imaging agent according to any of the preceding paragraphs.

In various embodiments the invention comprises using an agent that comprises an amplifier molecule that has the chemical structure of FIG. 3.

In various embodiments the invention comprises using an agent that comprises an amplifier molecule that has the chemical structure of FIG. 4. wherein RVP comprises the amino acid sequence CTPHTNQTC (SEQ ID NO: 1) and MC comprises a metal chelate according to any of the preceding paragraphs.

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

### EXAMPLES

### Example 1

In order to create an imaging or therapeutic agent a molecule was desired that would target vulnerable plaque. A rabbit model for vulnerable plaque in conjunction with phage display was utilized. In vivo phage display experiments were conducted, including both a C7×C and 7× library being injected into rabbits that had vulnerable plaque. For the first round of panning ˜1*10̂13 of phages from each library were injected into separate rabbits. The vulnerable plaque for each of these rabbits was collected and homogenized. The homogenate was treated with 50 mM glycine pH 2.2 to remove any bound phage before being amplified for the second round of injections into rabbits. After the second round the vulnerable plaque was excised and shipped in TBS. Both of these were treated with the pH 2.2 glycine buffer. The C7×C library was then amplified. The vulnerable plaque of the 7× library was treated with a 0.8 M sorbital 10 mM triethanolamine 1 mM EDTA pH 7.2 (cell lysis buffer) buffer and homogenized in a dounce homogenizer to lyse the cells and amplify phages that are within the cells and not on the surface. This procedure was repeated for the third round of panning. After this third round 20 isolated phages from both libraries were amplified and sequenced (Table 1).

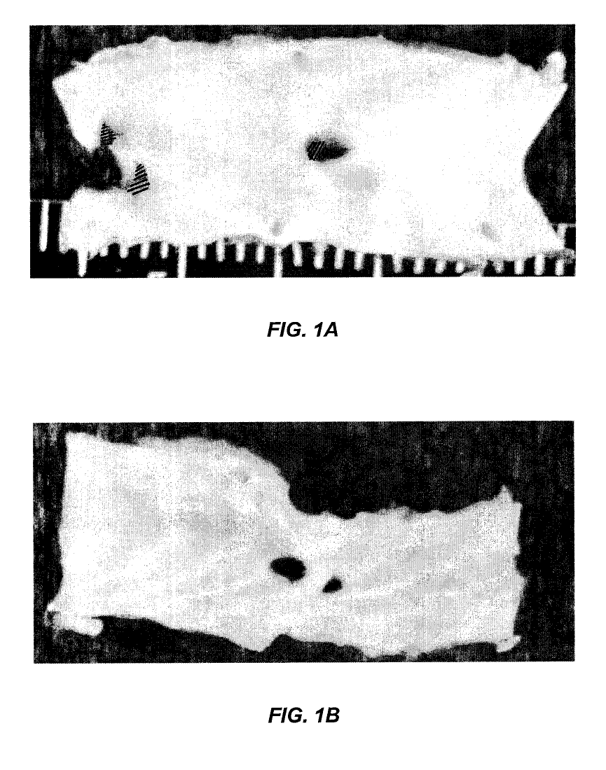

After the sequencing data was received the Rabbit Vulnerable Plaque (RVP) C7×C A peptide was selected for further testing. RVP has the sequence CTPHTNQTC (SEQ ID NO: 1) (in the one-letter code.) The clone was amplified in a 500 mL culture. Along with this an avidin-binding phage was also amplified and labeled as a negative control. Both of these phages were injected into separate vulnerable plaque rabbits. Afterwards the animals were sacrificed and the organs harvested. Aorta was washed then incubated with a biotinilated anti-phage mAb followed by a streptavadin-HRP. Sections of aorta were then imaged, FIG. 1A and FIG. 1B.

FIG. 1A shows clearly that phage bearing the peptide CTPHTNQTC (SEQ ID NO: 1) on their gIII tail proteins accumulate specifically at sites of plaque (crosshatching; compare with FIG. 1B.) Because of the method of selection of these peptides, the CTPHTNQTC (SEQ ID NO: 1) peptide may be internalized into cells in the plaque, providing a natural amplification mechanism for targeted imaging and therapy. The peptides discovered to bind vulnerable plaque and more fully described in Table 1 can be covalently attached to amplifier molecules. These compositions, now bearing multiple copies of the peptide can increase the amount of peptide bound to the atherosclerotic plaque and as a result, the amount of imaging or therapeutic agent bound to the plaque.

TABLE 1

Results from sequencing after the third round
of panning with rabbits targeting vulnerable
plaque. External phage was treated with glycine
to dissociate the phage and amplified. The
internalized phage were treated with glycine to
remove surface bound phage then treated with
triethanolamine to lyse cells to amplify only
internal phages. Of the 20 sequenced 10 of them
came back with the sequence CTPHTNQTC (SEQ ID
NO: 1). This was chosen as the most promising
candidate studies and is referred to as RVP
C7xC A.

| | | |
|---|---|---|
| | Peptide | Number of clones |
| Peptide name | sequence | with sequence |

Internalized phage

| | | |
|---|---|---|
| RVP C7xC A | CTPHTNQTC | 10 |
| | (SEQ ID NO: 1) | |
| RVP C7xC B | CWPRTFGAC | 3 |
| | (SEQ ID NO: 2) | |
| RVP C7xC C | CQATGKATC | 1 |
| | (SEQ ID NO: 3) | |
| RVP C7xC D | CTENARSQC | 1 |
| failed | (SEQ ID NO: 4) | 5 |

External phage

| | | |
|---|---|---|
| RVP C7xC E | CWNKSQMLC | 3 |
| | (SEQ ID NO: 5) | |
| RVP C7xC F | CHSNMRTSC | 1 |
| | (SEQ ID NO: 6) | |
| RVP C7xC G | CWPRTFGAC | 1 |
| | (SEQ ID NO: 2) | |
| RVP C7xC H | CDLLLSGNC | 1 |
| | (SEQ ID NO: 7) | |
| RVP C7xC I | CSSTNGNQC | 1 |
| | (SEQ ID NO: 8) | |
| RVP C7xC J | CTTSWIKNC | 1 |
| | (SEQ ID NO: 9) | |
| RVP C7xC K | CPTMSRPIC | 1 |
| | (SEQ ID NO: 10) | |
| RVP 7xA | SSLFPYN | 3 |
| | (SEQ ID NO: 11) | |
| RVP 7xB | NSSGRLP | 2 |
| | (SEQ ID NO: 12) | |
| RVP 7xC | SQHHWPV | 1 |
| | (SEQ ID NO: 13) | |
| RVP 7xD | SNASYQT | 1 |
| | (SEQ ID NO: 14) | |
| RVP 7xE | MTVAMTN | 1 |
| WT phage | (SEQ ID NO: 15) | 2 |
| Failed | | 1 |

1. A purified peptide that selectively binds to diseased tissue wherein said peptide comprises the amino acid sequence CTPHTNQTC (SEQ ID NO. 1).

2. The peptide of claim 1, wherein said diseased tissue is an atherosclerotic plaque.

3. The peptide of claim 1, wherein said diseased tissue is apoptotic.

4. The peptide of claim 1, wherein said peptide has the chemical structure:

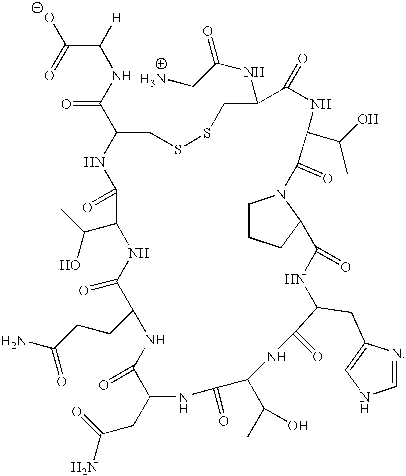

5. The peptide of claim 1, wherein said peptide has the formula:
X-(RVP)_{N}-M
wherein
X is an amplifier moiety;
RVP is a peptide comprising the amino acid sequence CTPHTNQTC; (SEQ ID NO. 1)
N is an integer selected from 2 to 100; and
M is an member selected from the group consisting of therapeutic agent and imaging agent.

6. The peptide of claim 5, wherein M is a metal chelate.

7. The peptide of claim 6, wherein the metal in said metal chelate is a member of the group consisting of Sn(II) and Sn(IV).

8. The peptide of claim 6, wherein the metal in said metal chelate is Sn-117m.

9. The peptide of claim 5, wherein said amplifier moiety comprises the chemical structure:

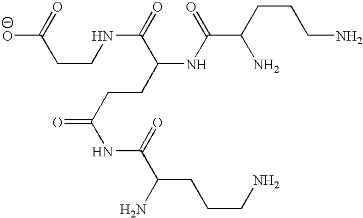

10. The peptide of claim 5, wherein said peptide comprises the chemical structure: 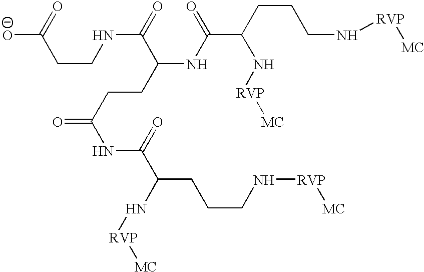
wherein MC comprises a methal chelate.

11. The peptide of claim 1, wherein said peptide binds to perilipin.

12. A pharmaceutical composition comprising the peptide of claim 1, and a pharmaceutically acceptable carrier.

13. A method of imaging atherosclerotic plaque in a subject, said method comprising:
(a) administering to said subject a purified peptide that selectively binds to atherosclerotic plaque wherein said peptide comprises the amino acid sequence CTPHTNQTC (SEQ ID NO. 1), wherein said peptide is covalently bound to an imaging agent, thereby forming with said atherosclerotic plaque an atherosclerotic plaque-peptide-agent complex; and
(b) detecting said atherosclerotic plaque-peptide-tin chelate covalent complex.

14. A method of treating atherosclerotic plaques in a subject, said method comprising:
(a) administering to said subject a purified peptide that selectively binds to atherosclerotic plaque wherein said peptide comprises the amino acid sequence CTPHTNQTC (SEQ ID NO. 1), wherein said peptide is covalently bound to an agent selected from the group consisting of therapeutic agent and imaging agent, thereby forming with said atherosclerotic plaque an atherosclerotic plaque-peptide-agent complex; and
(b) treating said atherosclerotic plaque.


SEQUENCE LISTING (15 sequences)

SEQ ID NO: 1 - protein, 9 aa, Homo sapiens
CTPHTNQTC

SEQ ID NO: 2 - protein, 9 aa, HOMO SAPIENS
CWPRTFGAC

SEQ ID NO: 3 - protein, 9 aa, HOMO SAPIENS
CQATGKATC

SEQ ID NO: 4 - protein, 9 aa, HOMO SAPIENS
CTENARSQC

SEQ ID NO: 5 - protein, 9 aa, HOMO SAPIENS
CWNKSQMLC

SEQ ID NO: 6 - protein, 9 aa, HOMO SAPIENS
CHSNMRTSC

SEQ ID NO: 7 - protein, 9 aa, HOMO SAPIENS
CDLLLSGNC

SEQ ID NO: 8 - protein, 9 aa, HOMO SAPIENS
CSSTNGNQC

SEQ ID NO: 9 - protein, 9 aa, HOMO SAPIENS
CTTSWIKNC

SEQ ID NO: 10 - protein, 9 aa, HOMO SAPIENS
CPTMSRPIC

SEQ ID NO: 11 - protein, 7 aa, HOMO SAPIENS
SSLFPYN

SEQ ID NO: 12 - protein, 7 aa, HOMO SAPIENS
NSSGRLP

SEQ ID NO: 13 - protein, 7 aa, HOMO SAPIENS
SQHHWPV

SEQ ID NO: 14 - protein, 7 aa, HOMO SAPIENS
SNASYQT

SEQ ID NO: 15 - protein, 7 aa, HOMO SAPIENS
MTVAMTN